(19) United States
(12) Patent Application Publication
(10) Pub. No.: US 2016/0377591 A1
(43) Pub. Date: Dec. 29, 2016

(54) DEVICES, SYSTEMS AND METHODS FOR SEQUENCING BIOMOLECULES

(71) Applicant: Quantum Biosystems Inc., Tokyo (JP)

(72) Inventors: Tomoji Kawai, Osaka (JP); Masateru Taniguchi, Osaka (JP); Takahito Ohshiro, Osaka (JP); Mark Floyd Oldham, Emerald Hills, CA (US); Eric Scott Nordman, Palo Alto, CA (US)

(21) Appl. No.: 15/217,821

(22) Filed: Jul. 22, 2016

Related U.S. Application Data
(63) Continuation of application No. PCT/JP2015/052601, filed on Jan. 23, 2015.

(30) Foreign Application Priority Data
Jan. 24, 2014 (JP) 2014-011430

(51) Int. Cl. G01N 33/487; G01N 27/447; C12Q 1/68

(52) U.S. Cl. CPC G01N 33/48721; C12Q 1/6869; G01N 27/4473

(57) ABSTRACT

The present disclosure provides a biomolecule sequencing device that includes at least one set of nano-gap electrodes arranged so that a current flows when a biomolecule contained in a sample passes in proximity to the set of nano-gap electrodes, an electrophoresis electrode pair for forming an electric field for moving the biomolecule between the electrodes of the set of nano-gap electrodes, a flow path for flowing the sample in a direction towards the nano-gap electrode pair, a flow path for flowing the sample in a direction away from the nano-gap electrode pair, a measurement unit configured to measure a tunnel current generated when the biomolecule passes between the electrodes of the nano-gap electrode pair with an electric field being formed, and an identification unit configured to sequence the biomolecule.

### TECHNICAL FIELD

The present invention relates to devices, methods and systems for sequencing biomolecules.

### BACKGROUND ART

Conventionally, sequencing has been used for determining the order of monomers that constitute a biological molecule, in particular, a biopolymer, for example, an amino-acid sequence constituting a protein, a nucleotide sequence constituting a nucleic acid, a monosaccharide sequence constituting a sugar chain, etc. For example, protein sequences have been determined using high performance liquid chromatography (HPLC), mass spectrometry, X-ray crystal structure analysis, Edman degradation, etc., which may be based on enzymatic decomposition.

### SUMMARY OF INVENTION

### Technical Problem

A single molecule electrical measurement method to identify single molecules using a tunnel current is a method that enables identification of single molecules by directly measuring the local density of states of a sample molecule. However, recognized herein are various limitations associated with such electrical measurement methods. With a method based on natural diffusion of a sample molecule, as in a sample molecule introduction method in conventional single molecule electrical measurement methods, most of the sample molecules may diffusionally change course or direction without passing through a sensing electrode in the middle of measurement of signals associated with the sample molecules. This may lead to erroneous results and inefficient sequencing. One issue is that long reads that may be necessary for sequencing biopolymers having a nucleic acid base, a sugar chain, a peptide chain, etc., are difficult to perform, such that sequence reading is restricted to short reads, and there are problems in that the frequency of passage of molecules between sensing electrodes is low, and therefore accuracy in molecule detection is low.

For introducing a sample molecule dissolved in a solvent, there are introduction methods using pumping pressure or electro-osmotic flow. However, none of these methods can induce a steady flow that can be controlled at the molecular scale. In electrophoresis controlled systems, molecules may move uniformly throughout a channel volume. Thus, simply increasing the frequency of passage of molecules between the sensing electrodes is insufficient. Thus, there is a disadvantage in utilizing some conventional single molecule electrical measurement methods using a tunneling current, as such a method is usable only in resequencing when and a high concentration pure sample solution is available.

The present disclosure provides devices, methods and systems for sequencing biomolecules that may solve various problems with methods and systems currently available. Methods and systems provide herein may enable the sequencing of biomolecules at a substantially high accuracy and high throughput compared to other methods and systems currently available. Methods and systems of the present disclosure can enable the sequencing of relatively high read lengths, which can provide a substantial enhancement of sequencing of other methods and systems.

### Solution to Problem

In an aspect, a biomolecule sequencing device includes at least one nano-gap electrode pair having electrodes arranged so that a tunnel current flows when one or more biomolecule(s) contained in a sample passes between the electrodes of a nano-gap electrode pair, the biomolecule being formed of connected single monomers of at least one kind; at least one electrophoresis electrode pair for forming an electrical field so as to move a biomolecule between the electrodes of the nano-gap electrode pair; a first flow path for flowing at least a part of a sample in a direction towards and between a nano-gap electrode pair in one or more nano-channels; and a second flow path for flowing the at least part of a sample in a direction past the entrance to one or more nano-channels containing one or more nano-gap electrode pairs. The biomolecule sequencing device may further include one or more measurement units configured to measure a tunnel current generated when a biomolecule passes between the electrodes of a nano-gap electrode pair through a first flow path utilizing an electrical field being formed so as to move said biomolecule in the moving direction by application of a voltage across an electrophoresis electrode pair; and an identification unit configured to identify at least one kind of monomer that comprise the biomolecule based on both a reference physical quantity of at least one kind of monomer of which the kind is known, and a detected physical quantity obtained from a tunnel current measured by the measurement unit.

According to the present invention, the biomolecule sequencing device may comprise one or more electrophoresis electrode pairs, a first flow path for flowing at least a part of the sample in a direction towards and between one or more nano-gap electrode pairs in a nano-channel, and a second flow path for flowing at least a part of the sample in a direction past the entrance to the nano-channel(s) containing one or more nano-gap electrode pairs. Accordingly, the efficiency of moving single molecules between the nano-gap electrode pairs can be improved as a result of an increase of the electric field impressed on a sample molecule. Furthermore, this enables identification of monomers with high accuracy and high throughput.

The biomolecule sequencing device may include a flow director configured to direct the flow of a sample, which may be a fluidic sample so that a first flow path and a second flow path may be formed, and so that fluidic communication may occur between a first path and a second path.

A flow director may be an insulator that extends towards an entrance to a nano-channel(s) with one or more nano-gap electrode pairs, within and electrically communicating with any fluid and other molecules contained in the nano-channel(s).

A nano-gap electrode pair and an electrophoresis electrode pair may be arranged in parallel to extend in a direction that intersects or is perpendicular to the direction of biomolecule movement. In an example, a nano-gap electrode and an electrophoresis electrode on each side of a channel are parallel to each other.

A nano-gap electrode pair may be disposed to extend in a direction that intersects the direction of movement of biomolecules in a nano-channel, and the electrophoresis electrode pair may be disposed on an insulator.

A long biomolecule may become self-entangled potentially causing clogging in the nanochannel or at the nanogap electrodes. Numerous pillars may be provided in the first flow path and the second flow path at intervals through which the biomolecules can pass, and which may be utilized for linearizing biomolecular polymers. In some embodiments, pillars may be provided within one or more nanochannels so as to linearize or maintain linearization of biomolecular polymers within a nano channel. For example, a single stranded DNA fragment may have a persistence length of 3 nanometers (nm) in 25 milli-mole (mM) NaCl, permitting significant structure to reform within a nanochannel with 100 nm minimum dimension for one or more of width, height or diameter, and such secondary structure may reform even in a nanochannel with a minimum feature size of 20nm or less, thus recreating a need to maintain linearization within a nanochannel.

There may be a plurality of nano-gap electrode pairs that may differ in inter-electrode distance.

The present invention also provides a biomolecule sequencing method that may be performed by a biomolecule sequencing device, the biomolecule sequencing device comprising: one or more nano-gap electrode pairs having electrodes arranged so that a tunnel current increases when a biomolecule contained in a sample passes between the electrodes, the biomolecule being formed of connected monomers of at least one kind; one or more electrophoresis electrode pairs for forming an electric field in a moving direction of a biomolecule moving between the electrodes of a nano-gap electrode pair; a first flow path for flowing at least a part of the sample in a direction towards and between a nano-gap electrode pair(s) in a nano-channel(s); and a second flow path for flowing at least a part of the sample in a direction away past the entrance to the nano-channel(s) containing at least one nano-gap electrode pair, the method including: measuring a tunnel current generated when a biomolecule passes between the electrodes of a nano-gap electrode pair through the first flow path with an electric field being formed so as to move the biomolecule by application of a voltage across the electrophoresis electrode pair; and identifying a kind of at least one kind of monomer that comprise the biomolecule based on both a reference physical quantity of at least one kind of monomer of which the kind is known and a detected physical quantity obtained from a tunnel current measured by the measurement unit.

The present invention also provides a biomolecule sequencing program that causes a computer to function as a measurement unit and an identification unit of the biomolecule sequencing device of the present invention.

According to the device, method, and program for sequencing biomolecules of the present invention, monomers constituting a biomolecule can be identified with high accuracy.

In another aspect, the present disclosure provides a biomolecule sequencing device, comprising: a nano-channel that permits a sample containing a biomolecule to move through the nano-channel; a plurality of sets of nano-gap electrodes in the nano-channel, wherein each set of the plurality of nano-gap electrodes is configured to permit the detection of a current when the biomolecule contained in the sample passes through the nano-channel and in proximity to the plurality of sets of nano-gap electrodes, and wherein at least two sets of the plurality of sets of nano-gap electrodes have different inter-electrode distances along a width of the nano-channel; and a set of electrophoresis electrodes that provide an electric field to subject the biomolecule to motion through the nano-channel and in proximity to the plurality of sets of nano-gap electrodes in the nano-channel.

In some embodiments of aspects provided herein, the biomolecule sequencing device further comprises: a measurement unit in communication with each of the plurality of sets of nano-gap electrodes, wherein the measurement unit is configured to measure the current generated when the biomolecule passes in proximity to the plurality of sets of nano-gap electrodes; and an identification unit in communication with the measurement unit, wherein the identification unit is configured to identify the biomolecule or a portion thereof.

In some embodiments of aspects provided herein, the biomolecule includes a plurality of monomers, and the identification unit is configured to identify the plurality of monomers based on a reference physical quantity of at least one known type of monomer and a physical quantity obtained from the current measured by the measurement unit. In some embodiments of aspects provided herein, the biomolecule sequencing device further comprises a flow director configured to generate a first flow path and a second flow path that are in fluid communication with the nano-channel, wherein the flow director directs a portion of the sample from the first flow path to the nano-channel and a remainder of the sample from the first flow path to the second flow path. In some embodiments of aspects provided herein, the flow director is an insulator that extends towards the plurality of sets of nano-gap electrodes along a direction of movement of the sample through the nano-channel. In some embodiments of aspects provided herein, the biomolecule sequencing device further comprises one or more pillars in the first path and/or second flow path to permit linearization of the biomolecule. In some embodiments of aspects provided herein, the one or more pillars includes a plurality of pillars. In some embodiments of aspects provided herein, the first flow path, the second flow path and the nano-channel are substantially in the same plane. In some embodiments of aspects provided herein, the current includes tunneling current. In some embodiments of aspects provided herein, a given set of the plurality of sets of nano-gap electrodes has at least two electrodes. In some embodiments of aspects provided herein, the set of electrophoresis electrodes has at least two electrodes. In some embodiments of aspects provided herein, the plurality of sets of nano-gap electrodes and the set of electrophoresis electrodes are integrated as a single-piece unit. In some embodiments of aspects provided herein, electrodes of a given set of the plurality of sets of nano-gap electrodes are separated from the electrophoresis electrodes by at least one solid state insulator. In some embodiments of aspects provided herein, the biomolecule sequencing device further comprises one or more pillars in the nano-channel to permit linearization of the biomolecule. In some embodiments of aspects provided herein, the one or more pillars includes a plurality of pillars. In some embodiments of aspects provided herein, the nano-channel is tapered towards the plurality of sets of nano-gap electrodes. In some embodiments of aspects provided herein, a given set of the plurality of sets of nano-gap electrodes has an inter-electrode distance that is less than or equal to a molecular diameter of the biomolecule.

Another aspect of the present disclosure provides a biomolecule sequencing device, comprising: a nano-channel that permits a sample containing a biomolecule to move through the nano-channel; at least one set of nano-gap electrodes in the nano-channel, wherein the set of nano-gap electrodes is configured to permit the detection of a current when the biomolecule contained in the sample passes through the nano-channel and in proximity to the set of nano-gap electrodes, wherein the nano-channel is tapered towards the set of nano-gap electrodes, and wherein the set of nano-gap electrodes has an inter-electrode distance that is less than or equal to a molecular diameter of the biomolecule; and a set of electrophoresis electrodes that provide an electric field to subject the biomolecule to motion through the nano-channel and in proximity to the set of nano-gap electrodes in the nano-channel.

Another aspect of the present disclosure provides a biomolecule sequencing device, comprising: a nano-channel that permits a sample containing a biomolecule to move through the nano-channel; at least one set of nano-gap electrodes in the nano-channel, wherein the set of nano-gap electrodes is configured to permit the detection of a current when the biomolecule contained in the sample passes through the nano-channel and in proximity to the set of nano-gap electrodes; a set of electrophoresis electrodes that provide an electric field to subject the biomolecule to motion through the nano-channel and in proximity to the set of nano-gap electrodes in the nano-channel; and one or more pillars in or in proximity to the nano-channel, wherein the one or more pillars linearize the biomolecule to permit identification of individual subunits of the biomolecule using the current detection by the set of nano-gap electrodes.

Another aspect of the present disclosure provides a method for sequencing a biomolecule, comprising: (a) directing the biomolecule to flow to or through a nano-channel of a biomolecule sequencing device, wherein the biomolecule sequencing device includes (i) a plurality of sets of nano-gap electrodes in the nano-channel, wherein each set of the plurality of nano-gap electrodes is configured to permit the detection of a current when the biomolecule contained in the sample passes through the nano-channel and in proximity to the plurality of sets of nano-gap electrodes, and wherein at least two sets of the plurality of sets of nano-gap electrodes have different inter-electrode distances along a width of the nano-channel, and (ii) a set of electrophoresis electrodes that provide an electric field to subject the biomolecule to motion to or through the nano-channel and in proximity to the plurality of sets of nano-gap electrodes in the nano-channel; (b) with the plurality of sets of nano-gap electrodes, detecting current generated while the biomolecule flows through the nano-channel and in proximity to the plurality of sets of nano-gap electrodes; and (c) sequencing the biomolecule or a portion thereof with the current detected in (b).

In some embodiments of aspects provided herein, the biomolecule includes a plurality of monomers, and the sequencing comprises identifying the plurality of monomers based on a reference physical quantity of at least one known type of monomer and a physical quantity obtained from the current detected in (b). In some embodiments of aspects provided herein, the biomolecule sequencing device further comprises a flow director configured to generate a first flow path and a second flow path that are in fluid communication with the nano-channel, and (a) comprises flowing a portion of the sample from the first flow path to the nano-channel and a remainder of the sample from the first flow path to the second flow path. In some embodiments of aspects provided herein, the method further comprises one or more pillars in the first path and/or second flow path to permit linearization of the biomolecule. In some embodiments of aspects provided herein, the current includes tunneling current. In some embodiments of aspects provided herein, the method further comprises one or more pillars in the nano-channel that linearize the biomolecule. In some embodiments of aspects provided herein, the nano-channel is tapered towards the plurality of sets of nano-gap electrodes. In some embodiments of aspects provided herein, the biomolecule is a polynucleotide or a polypeptide.

Another aspect of the present disclosure provides a method for sequencing a biomolecule, comprising: (a) directing the biomolecule to flow to or through a nano-channel of a biomolecule sequencing device, wherein the biomolecule sequencing device includes (i) at least one set nano-gap electrodes in the nano-channel, wherein the set of nano-gap electrodes is configured to permit the detection of a current when the biomolecule contained in the sample passes through the nano-channel and in proximity to the set of nano-gap electrodes, wherein the nano-channel is tapered towards the set of nano-gap electrodes, wherein the set of nano-gap electrodes has an inter-electrode distance that is less than or equal to a molecular diameter of the biomolecule, and (ii) a set of electrophoresis electrodes that provide an electric field to subject the biomolecule to motion to or through the nano-channel and in proximity to the set of nano-gap electrodes in the nano-channel; (b) with the set of nano-gap electrodes, detecting current generated while the biomolecule flows through the nano-channel and in proximity to the set of nano-gap electrodes; and (c) sequencing the biomolecule or a portion thereof with the current detected in (b).

Another aspect of the present disclosure provides a method for sequencing a biomolecule, comprising: (a) directing the biomolecule to flow to or through a nano-channel of a biomolecule sequencing device, wherein the biomolecule sequencing device includes (i) at least one set nano-gap electrodes in the nano-channel, wherein the set of nano-gap electrodes is configured to permit the detection of a current when the biomolecule contained in the sample passes through the nano-channel and in proximity to the set of nano-gap electrodes, (ii) a set of electrophoresis electrodes that provide an electric field to subject the biomolecule to motion to or through the nano-channel and in proximity to the set of nano-gap electrodes in the nano-channel, and (iii) one or more pillars in or in proximity to the nano-channel, wherein the one or more pillars linearize the biomolecule to permit identification of individual subunits of the biomolecule using the current detection by the set of nano-gap electrodes; (b) with the set of nano-gap electrodes, detecting current generated while the biomolecule flows through the nano-channel and in proximity to the set of nano-gap electrodes; and (c) sequencing the biomolecule or a portion thereof with the current detected in (b).

Another aspect of the present disclosure provides a computer readable medium comprising machine executable code that upon execution by one or more computer processors implements a method for sequencing a biomolecule, comprising: (a) directing the biomolecule to flow to or through a nano-channel of a biomolecule sequencing device, wherein the biomolecule sequencing device includes (i) a plurality of sets of nano-gap electrodes in the nano-channel, wherein each set of the plurality of nano-gap electrodes is configured to permit the detection of a current when the biomolecule contained in the sample passes through the nano-channel and in proximity to the plurality of sets of nano-gap electrodes, and wherein at least two sets of the plurality of sets of nano-gap electrodes have different inter-electrode distances along a width of the nano-channel, and (ii) a set of electrophoresis electrodes that provide an electric field to subject the biomolecule to motion to or through the nano-channel and in proximity to the plurality of sets of nano-gap electrodes in the nano-channel; (b) with the plurality of sets of nano-gap electrodes, detecting current generated while the biomolecule flows through the nano-channel and in proximity to the plurality of sets of nano-gap electrodes; and (c) sequencing the biomolecule or a portion thereof with the current detected in (b).

Another aspect of the present disclosure provides a computer readable medium comprising machine executable code that upon execution by one or more computer processors implements a method for sequencing a biomolecule, comprising: (a) directing the biomolecule to flow to or through a nano-channel of a biomolecule sequencing device, wherein the biomolecule sequencing device includes (i) at least one set nano-gap electrodes in the nano-channel, wherein the set of nano-gap electrodes is configured to permit the detection of a current when the biomolecule contained in the sample passes through the nano-channel and in proximity to the set of nano-gap electrodes, wherein the nano-channel is tapered towards the set of nano-gap electrodes, wherein the set of nano-gap electrodes has an inter-electrode distance that is less than or equal to a molecular diameter of the biomolecule, and (ii) a set of electrophoresis electrodes that provide an electric field to subject the biomolecule to motion to or through the nano-channel and in proximity to the set of nano-gap electrodes in the nano-channel; (b) with the set of nano-gap electrodes, detecting current generated while the biomolecule flows through the nano-channel and in proximity to the set of nano-gap electrodes; and (c) sequencing the biomolecule or a portion thereof with the current detected in (b).

Another aspect of the present disclosure provides a computer readable medium comprising machine executable code that upon execution by one or more computer processors implements a method for sequencing a biomolecule, comprising: (a) directing the biomolecule to flow to or through a nano-channel of a biomolecule sequencing device, wherein the biomolecule sequencing device includes (i) at least one set nano-gap electrodes in the nano-channel, wherein the set of nano-gap electrodes is configured to permit the detection of a current when the biomolecule contained in the sample passes through the nano-channel and in proximity to the set of nano-gap electrodes, (ii) a set of electrophoresis electrodes that provide an electric field to subject the biomolecule to motion to or through the nano-channel and in proximity to the set of nano-gap electrodes in the nano-channel, and (iii) one or more pillars in or in proximity to the nano-channel, wherein the one or more pillars linearize the biomolecule to permit identification of individual subunits of the biomolecule using the current detection by the set of nano-gap electrodes; (b) with the set of nano-gap electrodes, detecting current generated while the biomolecule flows through the nano-channel and in proximity to the set of nano-gap electrodes; and (c) sequencing the biomolecule or a portion thereof with the current detected in (b).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 is a schematic view illustrating a biomolecule sequencing device.

FIG. 2 is an enlarged view showing a top view of a nano-gap electrode pair of FIG. 1.

FIG. 3 is an enlarged view showing a part of FIG. 2.

FIG. 4 is a block diagram illustrating a functional configuration of a control unit.

FIG. 5 is a flowchart showing a biomolecule sequencing process.

FIG. 6 is a data showing a waveform of a signal detected when an electrophoresis electrode pair is not provided.

FIG. 7 is a data showing a waveform of a signal detected when the electrophoresis electrode pair is provided.

FIG. 8 is a graph showing a signal frequency.

FIG. 9 is a graphic representation showing the number of reads when an electrophoresis electrode pair is provided and utilized and when an electrophoresis electrode pair is not provided.

FIG. 10 is a graphic representation of the number of reads per unit time when an electrophoresis electrode pair is provided and utilized and when an electrophoresis electrode pair is not provided.

FIG. 11 illustrates a variant example of an arrangement of an electrophoresis electrode pair.

FIG. 12 is a schematic view illustrating a structure of a biomolecule sequencing device with variable spaced nano-gaps.

FIG. 13 is a block diagram illustrating a flowchart of a functional configuration of a control unit useable with variably spaced nano-gaps.

FIG. 14 is a flowchart showing a biomolecule sequencing process useable with variably spaced nano-gaps.

FIG. 15 schematically illustrates a computer control system that is programmed or otherwise configured to implements devices, systems and methods of the present disclosure.

### DESCRIPTION OF EMBODIMENTS

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “gap,” as used herein, generally refers to a break or hole in an object or between two objects. The object may be a solid state object, such as a substrate or an electrode. The gap may be disposed adjacent or in proximity to a sensing circuit or an electrode coupled to a sensing circuit. In some examples, a gap has a characteristic width or diameter on the order of 0.1 nanometers (nm) to about 1000 nm. A gap having a width on the order of nanometers is referred to as a “nanogap” or “nano-gap.” In some situations, a nano-gap has a width that is from about 0.1 nanometers (nm) to 50 nm, 0.5 nm to 30 nm, or 0.5 nm or 10 nm, 0.5 nm to 5 nm, or 0.5 nm to 2 nm, or no greater than 2 nm, 1 nm, 0.9 nm, 0.8 nm, 0.7 nm, 0.6 nm, or 0.5 nm. In some cases, a nano-gap has a width that is at least about 0.5 nm, 0.6 nm, 0.7 nm, 0.8 nm, 0.9 nm, 1 nm, 2 nm, 3 nm, 4 nm, or 5 nm. In some cases, the width of a nano-gap can be less than or equal to a molecular diameter (e.g., average molecular diameter) of a biomolecule or a subunit (e.g., monomer) of the biomolecule.

The term “channel,” as used herein, generally refers to a pore, passage or conduit formed or otherwise provided in a material. The material may be a solid state material, such as a substrate. In some examples, a channel has a characteristic width or diameter on the order of 0.1 nanometers (nm) to about 1000 nm. A channel having a width on the order of nanometers is referred to as a “nanochannel” or “nano-channel.” In some situations, a nano-channel has a width that is from about 0.1 nanometers (nm) to 50 nm, 0.5 nm to 30 nm, or 0.5 nm or 10 nm, 0.5 nm to 5 nm, or 0.5 nm to 2 nm, or no greater than 2 nm, 1 nm, 0.9 nm, 0.8 nm, 0.7 nm, 0.6 nm, or 0.5 nm. In some cases, a nano-channel has a width that is at least about 0.5 nm, 0.6 nm, 0.7 nm, 0.8 nm, 0.9 nm, 1 nm, 2 nm, 3 nm, 4 nm, or 5 nm. In some cases, the width of a nano-channel or a portion of the nano-channel (e.g., tapered portion of the nano-channel) can be less than or equal to a molecular diameter (e.g., average molecular diameter) of a biomolecule or a subunit (e.g., monomer) of the biomolecule.

The term “current,” as used herein, generally refers to electrical current. Current that is on the order of micro or nano amperes may be referred to as a “nano current” (also “nanocurrent” herein). In some examples, current is or includes tunneling current.

The term “electrode,” as used herein, generally refers to a material that can be used to measure electrical current. An electrode can be used to measure electrical current to or from another electrode. In some situations, electrodes can be disposed in a channel (e.g., nanogap) and be used to measure the current across the channel. The current can be a tunneling current. Such a current can be detected upon the flow of a biomolecule (e.g., protein) through the nanogap. In some cases, a sensing circuit coupled to electrodes provides an applied voltage across the electrodes to generate a current. As an alternative or in addition to, the electrodes can be used to measure and/or identify the electric conductance associated with a biomolecule (e.g., an amino acid subunit or monomer of a protein). In such a case, the tunneling current can be related to the electric conductance.

The term “biomolecule,” as used herein generally refers to any biological material that can be interrogated with an electrical current and/or potential across a nano-gap electrode. A biomolecule can be a nucleic acid molecule, protein, or carbohydrate. A biomolecule can include one or more subunits, such as nucleotides or amino acids. A biomolecule can be deoxyribonucleic acid (DNA) or ribonucleic acid (RNA), or a derivative thereof. A biomolecule can be a fragment of a larger molecule, such as a DNA fragment of a larger DNA sample.

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is DNA or RNA, or derivatives thereof. A nucleic acid may be single-stranded or double stranded.

The term “protein,” as used herein, generally refers to a biological molecule, or macromolecule, having one or more amino acid monomers, subunits or residues. A protein containing 50 or fewer amino acids, for example, may be referred to as a “peptide.” The amino acid monomers can be selected from any naturally occurring and/or synthesized amino acid monomer, such as, for example, 20, 21, or 22 naturally occurring amino acids. In some cases, 20 amino acids are encoded in the genetic code of a subject. Some proteins may include amino acids selected from about 500 naturally and non-naturally occurring amino acids. In some situations, a protein can include one or more amino acids selected from isoleucine, leucine, lysine, methionine, phenylalanine, threonine, tryptophan and valine, arginine, histidine, alanine, asparagine, aspartic acid, cysteine, glutamine, glutamic acid, glycine, proline, serin and tyrosine.

The term “set,” as used herein, generally refers to a group or collection of elements. A set can include a plurality of elements. A set can include a “pair,” or two. For example, a set of electrodes can include at least 2, 3, 4, 5, 6, 7, 8, 9, or 10 electrodes.

The term “sequencing,” as used herein, generally refers to methods and technologies for determining the sequence of a biomolecule, such as the sequence of nucleotide bases in one or more polynucleotides, or the sequence of amino acids in a polypeptide.

The term “read,” as used herein, generally refers to a sequence of a biomolecule or a portion of the biomolecule as generated by a sequencing device or system. Such sequence may be of sufficient length (e.g., at least about 30 base pairs (bp)) that can be used to identify a larger sequence or region, e.g., that can be aligned to a location on a chromosome or genomic region or gene.

### Sequencing Devices and Systems

The present disclosure provides devices for sequencing biomolecules. A sequencing device can include at least 1, 2, 3, 4, 5, 6, 7, 8, 9, 10, 100, 1000, or 10000 channels. A channel can include at least 1, 2, 3, 4, 5, 6, 7, 8, 9, or 10 sets of nano-gap electrodes in the channel. The channel can be a nano-channel. Electrodes of a set of nano-gap electrodes can be oppositely situated in the channel.

A biomolecule (e.g., single-stranded DNA or RNA, double-stranded DNA or RNA, or a protein) can be subjected to flow in or through the channel and a current, in some cases a tunneling current, can be measured across the channel using electrodes of a given set of nano-gap electrodes. The current can be or include tunneling current. The biomolecule can be subjected to flow in or through the channel using an electric field provided by at least 1, 2, 3, 4, 5, 6, 7, 8, 9, or 10 set of electrophoresis electrodes.

The channel can be part of a nanopore. The nanopore can be formed in a membrane, such as a solid state membrane.

A set of nano-gap electrodes can be configured to detect a current when a biomolecule passes through the channel and in proximity to the set of nano-gap electrodes. The set of nano-gap electrodes can have different inter-electrode distances.

A set of electrophoresis electrodes can provide an electric field to subject the biomolecule to motion through the nano-channel and in proximity to the plurality of sets of nano-gap electrodes in the nano-channel. The electric field can be generated upon application of a voltage or voltage pulse to the electrophoresis electrodes. In some examples, the electric field has a strength from about 0.1 Newtons (N) per coulomb (C) to 5000 N/C, or from 1 N/C to 250 N/C, or from 10 N/C to 50 N/C.

The set of electrophoresis electrodes can be situated external to the channel. Alternatively, the set of electrophoresis electrodes and the set of nano-gap electrodes can be integrated as a single-piece unit. For example, an electrode of the nano-gap electrodes can be separated from an electrophoresis electrode among the set of electrophoresis electrodes by at least one solid state insulator.

The biomolecule can be identified or sequenced using a computer control unit. The computer control unit can be part of the sequencing device or a sequencing system that includes the sequencing device. The computer control unit can include a measurement unit in communication with the set of nano-gap electrodes. The measurement unit is configured to measure the current generated when the biomolecule passes in proximity to the plurality of sets of nano-gap electrodes. The computer control unit can further include an identification unit in communication with the measurement unit. The identification unit is configured to identify the biornolecule or a portion thereof.

In some cases, the biomolecule includes a plurality of monomers (or subunits). The identification unit can be configured to identify the plurality of monomers based on a reference physical quantity of at least one known type of monomer and a physical quantity obtained from the current measured by the measurement unit.

The sequencing device can include a flow director configured to generate at least a first flow path and a second flow path that are in fluid communication with the channel. The flow director can direct a portion of the sample from the first flow path to the channel and a remainder of the sample from the first flow path to the second flow path. The flow director can be an insulator that extends towards the set of nano-gap electrodes along a direction of movement of the sample through the nano-channel.

The sequencing device can include at least 1, 2, 3, 4, 5, 6, 7, 8, 9, 10, or 100 pillars. A pillar can be in the channel or external to the channel, such as in the first path and/or second flow path. The pillar can permit linearization of the biomolecule, which can aid in effectively sequencing or identifying the biomolecule or portion (e.g., subunit) thereof.

The first flow path, the second flow path and the nano-channel can be substantially in the same plane (i.e., coplanar). As an alternative, the first flow path, the second flow path and the nano-channel are not in the same plane.

The channel can include at least 1, 2, 3, 4, 5, 6, 7, 8, 9, or 10 tapered portions. Such tapering can be at a portion of the channel that is at or adjacent to a set of nano-gap electrodes.

The set of nano-gap electrodes can include at least 2, 3, 4, 5, 6, 7, 8, 9, or 10 electrodes. The electrodes can have an inter-electrode spacing (or distance) from 0.1 nanometers (nm) to 50 nm, 0.5 nm to 30 nm, or 0.5 nm or 10 nm, 0.5 nm to 5 nm, or 0.5 nm to 2 nm, or no greater than 2 nm, 1 nm, 0.9 nm, 0.8 nm, 0.7 nm, 0.6 nm, or 0.5 nm. In some cases, the spacing is at least about 0.5 nm, 0.6 nm, 0.7 nm, 0.8 nm, 0.9 nm, 1 nm, 2 nm, 3 nm, 4 nm, or 5 nm. In some examples, the spacing is less than or equal to a molecular diameter of the biomolecule.

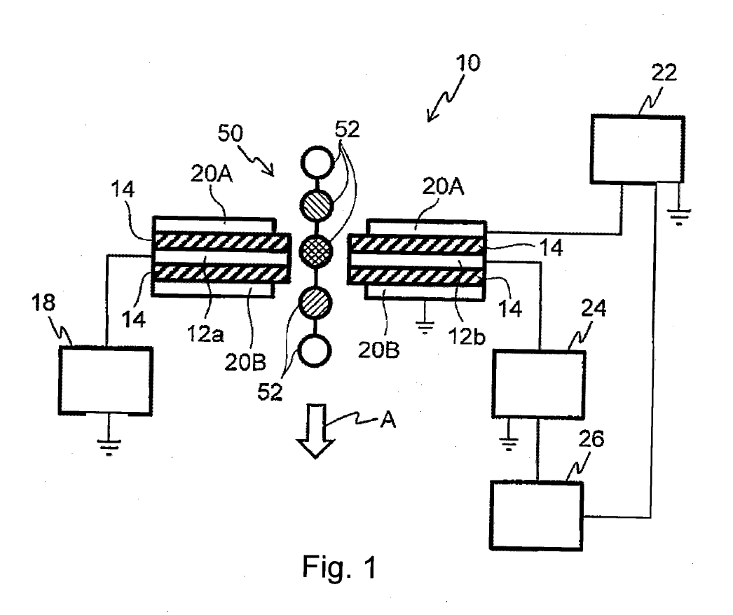

FIG. 1 shows a biomolecule sequencing device 10 in some embodiments may include a nano-gap electrode pair 12 (12A and 12B), a measurement power supply device 18, an electrode pair for electrophoresis (hereinafter, referred to as “electrophoresis electrode pair”) 20 (20A and 20B), a power supply device 22 for electrophoresis, an ammeter 24, and a system control unit 26. Each of the components will be described below.

Nano-gap electrode pair 12 may comprise a pair of opposed nano-gap electrodes 12a and 12b. Nano-gap electrodes 12a and 12b may be arranged at a distance such that a tunnel current flow therebetween increases when a monomer 52 of a biomolecule contained in a sample 50 passes between the electrodes. Here, the biomolecules include proteins, peptides, nucleic acids, sugar chains, etc. Monomers comprising a biomolecule may include, but are not limited to, amino acids comprising a protein or a peptide, nucleotides comprising a nucleic acid, monosaccharide comprising a polysaccharide or sugar chain, etc.

While a pair of nano-gap electrodes 12a and 12b are shown and described, the device 10 can include more than two electrodes. For example, the device 10 can include a set of nano-gap electrodes, with the set including at least 2, 3, 4, 5, 6, 7, 8, 9, or 10 electrodes.

When an inter-electrode distance is much longer than a molecular diameter of a single molecule 52, a tunnel current may not readily flow between the electrodes of a nano-gap electrode pair 12, or two or more single molecules 52 may enter between a nano-gap electrode pair 12 at the same time. In contrast, when an inter-electrode distance is much shorter than a molecular diameter of a single molecule 52, a single molecule 52 cannot enter between the electrodes of a nano-gap electrode pair 12.

When an inter-electrode distance is much longer or much shorter than a molecular diameter of a single molecule 52, detecting a tunnel current passing through a single molecule 52 may be difficult. Thus, an inter-electrode distance may be preferably made slightly shorter than, or the same as, or slightly longer than, a molecular diameter of a single molecule 52. For example, an inter-electrode distance may be a length that is 0.5 times to 2 times the molecular diameter of a single molecule 52, with an inter-electrode distance optionally being set at a length of 0.5 to 1 times a molecular diameter, and optionally being set at a length 0.7 to 0.9 times the molecular diameter.

Specific methods for fabricating a nano-gap electrode 12 are not particularly limited. An example of such a fabrication method will be described below.

A pair of nano-gap electrodes 12 mentioned above can be fabricated using a known nanofabricated mechanically controllable break junction method. A nanofabricated mechanically controllable break junction method is an excellent method that is capable of controlling an inter-electrode distance with excellent mechanical stability at a picometer-level or finer resolution. Fabrication methods for electrode pairs using nanofabricated mechanically controllable break junction methods are described, for example, in T. M. van Ruitenbeek, A. Alvarez, I. Pineyro, C. Grahmann, P. Joyez, M. H. Devoret, D. Esteve, C. Urbina, Rev. Sci. Instrum. 67, 108 (1996), and M. Tsutsui, K. Shoji, M. Taniguchi, T. Kawai, Nano Lett. 8, 345 (2008). Electrode materials include any appropriate metal such as gold, platinum, silver, palladium, tungsten, and appropriate alloys or composites.

For example, a nano-gap electrode pair 12 may be fabricated using the following procedure.

First, using electron beam lithography and lift-off technology, nanoscale gold junctions may be patterned on a polyimide coated flexible metal substrate using an electron beam lithography device (e.g., JEOL Ltd., catalogue number: JSM6500F). Then, polyimide under junctions may be removed by etching using an etching process, e.g., a reactive ion etching process, which can be performed, for example, by using a reactive ion etching device (e.g., Samco Inc., catalogue number: 10NR).

After this, a nanoscale gold bridge structure with a 3-point bent structure may be fabricated by bending a substrate. At this time, an inter-electrode distance of an electrode pair can be controlled at a picometer-level or finer resolution by controlling precise bending of the substrate using a piezoelectric actuator (CEDRAT, catalogue number: APA150M).

In some embodiments, by using such fabrication methods and processes, a device which is substantially planar may be effectuated. One or nano-channels may be fabricated such that the nano-channel(s) may be fabricated on or above a substrate. Middle region(s) 44M, at the ends of the one or more nano-channel, may be situated such the bottom of the middle region(s) 44M may be at the same, or substantially the same vertical distance above a substrate as the bottom of the one or more nano-channels wherein the ends of said one or more nano-channels are immediately adjacent to said middle regions.

In further embodiments, middle region(s) 44M, at the ends of the one or more nano-channel, may be situated such the top of the middle region(s) 44M may be at the same, or substantially the same vertical distance above a substrate as the top of the one or more nano-channels wherein the ends of said one or more nano-channels are immediately adjacent to said middle regions.

In some embodiments, a vertical dimension may be the same or substantially the same, or coplanar if it is within the fabrication tolerances that may otherwise permit the dimension to be exactly the same.

In other embodiments, one or more nano-channels may be considered to have a vertical dimension which may be considered to be the same or substantially the same, or coplanar if the open ends of the nano-channel intersect the middle region(s) at the ends of the one or more nano-channels, wherein the entire vertical dimension of the nano-channel is contained within the vertical dimensions of the middle regions.

In further embodiments, one or more nano-channels may be considered to have a vertical dimension which may be considered to be the same or substantially the same, or coplanar if the open ends of the nano-channel intersect the middle region(s) at the ends of the one or more nano-channels, wherein at least a half of the vertical dimension of the nano-channel is contained within the vertical dimensions of the middle regions.

As a result, a bridge so provided may be pulled so that the bridge is partly broken. The bridge may be further pulled, and the size of a nano-gap (inter-electrode distance) occurring due to a break may be set to a desired length corresponding to detection of a target single molecule 52. For example, if a single molecule 52 is an amino acid molecule that constitutes a peptide obtained by cleaving a protein, which is a biomolecule, into a certain length, the length of a side chain of a monomer of a the single molecule 52, may be about 0.3 nm to 1 nm. In this case, an inter-electrode distance of the electrode pair may be accurately controlled by regulating bridge pulling using a self-breaking technology (see for example M. Tsutsui, K. Shoji, M. Taniguchi, T. Kawai, Nano Lett. 8, 345 (2008) and M. Tsutsui, M. Taniguchi, T. Kawai, Appl. Phys. Lett. 93, 163115 (2008)).

Specifically, a DC bias voltage (Vb) of 0.1 V, or 0.050 V to 0.4 V may be applied to a bridge using a series resistance of 10 kΩ and a gold nanojunction pulled at a programmed junction stretching speed, thereby breaking the bridge, utilizing a resistance feedback method (see M. Tsutsui, K. Shoji, M. Taniguchi, T. Kawai, Nano Lett. 8, 345 (2008), and M. Tsutsui, M. Taniguchi, T. Kawai, Appl. Phys. Lett. 93, 163115 (2008)) for example employing a data acquisition board (National Instruments Corporation, catalogue number: NI PCIe-6321). A bridge may be further pulled, and a size of a nano-gap (inter-electrode distance) occurring due to a break may be set to an intended length. Thus, a nano-gap electrode pair 12 may be formed.

A voltage may be applied across a nano-gap electrode pair 12 by a measurement power supply device 18. A voltage that may be applied to a nano-gap electrode pair 12 by a measurement power supply device 18 is not particularly limited, and may be, for example, 0.25V to 0.75V, or 0.1V to 0.4V, or 0.050V to 0.02V. There is no particular limitation to a specific configuration of a measurement power supply device 18, and an appropriate known power supply device may be used.

An electrophoresis electrode pair 20 may comprise a pair of electrophoresis electrodes 20A and 20B. Electrophoresis electrodes 20A and 20B may be arranged so that an electric field may be formed in a direction such that a single molecule 52 contained in a sample 50 may be moved (the direction indicated by block arrow A in FIG. 1). In some embodiments, by way of example, electrophoresis electrodes 20A and 20B may be placed such that a sample molecule may be moved relative to a nano-gap electrode pair 12 with insulators 14 being sandwiched therebetween. The width of the insulator 14 may be set to a width (for example, about 300 nm) that is sufficient so that no interference occurs between a current flowing across the electrophoresis electrode pair 20 and a current flowing across a nano-gap electrode pair 12.

In the example in FIG. 1, electrophoresis electrode 20A may be composed of two separated electrodes, but it need not be separated and may be a single electrode. This may also apply to electrophoresis electrodes 20B.

When an electric field is formed between electrophoresis electrode 20A and electrophoresis electrode 20B, a single molecule 52 may be moved by the electric field by electrophoresis and or electroosmosis. In other words, single molecule 52 may move so as to pass between the electrodes of nano-gap electrode pair 12.

A voltage may be applied across electrophoresis electrode pair 20 by an electrophoresis power supply device 22. A voltage that is applied to electrophoresis electrode pair 20 by electrophoresis power supply device 22 is not particularly limited, and a voltage capable of controlling a speed at which single molecule 52 passes between the electrodes of nano-gap electrode pair 12 may be appropriately set. Electrophoresis power supply 22 may apply a voltage to electrophoresis electrode pair 20 so that the direction of the electric field formed between the electrodes of electrophoresis electrode pair 20 may be switched. Thus, direction of movement of single molecule 52 moving between the electrodes of electrophoresis electrode pair 20 can be switched. There is no particular limitation to a specific configuration of the electrophoresis power supply device 22, and an appropriate known power supply device may be used.

Ammeter 24 may measure an increase in tunneling current that is generated when a monomer 52 passes between the electrodes of a nano-gap electrode pair 12 across which a voltage is applied by measurement power supply device 18. There is no particular limitation to a specific configuration of ammeter 24, and an appropriate known current measurement device such as a transimpedance amplifier may be used.

Next, a specific configuration associated with a nano-gap electrode pair 12 and an electrophoresis electrode pair 20 of a biomolecule sequencing device 10 will be described.

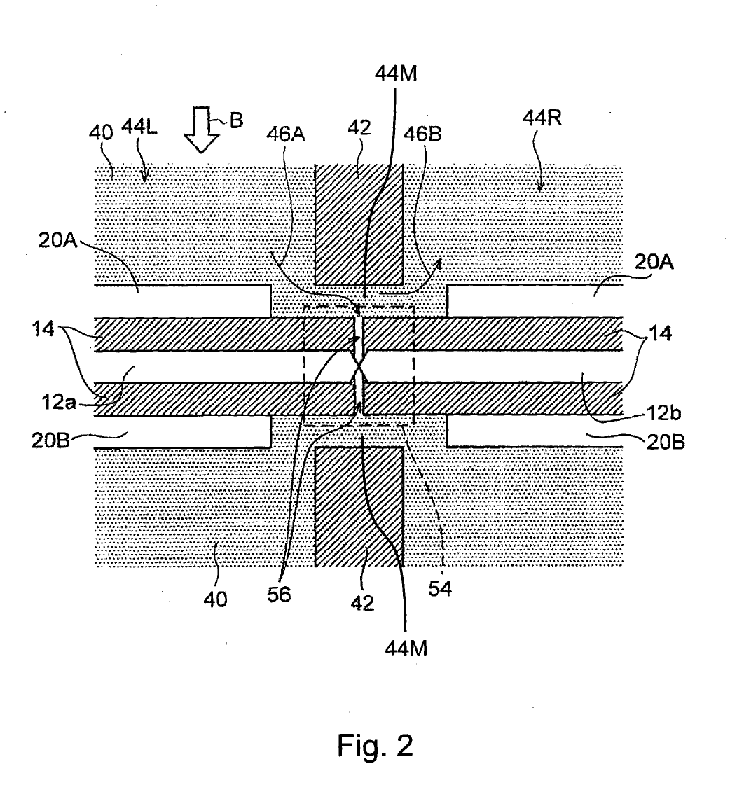

FIG. 2 is an enlarged view showing the periphery of a nano-gap electrode pair 12 and an electrophoresis electrode pair 20. As illustrated in FIG. 2, numerous nano-pillars 40 may be provided at intervals so that a single molecule 52 can pass around the nano-pillars to get to a nano-gap electrode pair(s) 12 and electrophoresis electrode pair 20. As used herein, a “nano pillar” may be a pillar on the scale of a nanometer or less in diameter or width.

A sample 50 may be guided from a left region 44L wherein nano-pillars 40 may be provided, which can be seen in the upper left of FIG. 2, in the region indicated by arrow B. A sample 50 may be moved by one of electrophoresis, electroosmosis, pressure, surface tension, diffusion and combinations thereof. Complicated entangling biomolecules such as DNA, etc., contained in a sample 50 may be separated from other DNA molecules, detangled or linearized by the great number of nano-pillars 40 that are arranged like stalks of bamboo in a grove.

In some embodiments, a sample, which may be a fluidic sample, may be introduced into a device in a manner such that capillary action may cause said sample to be drawn, for example from a left region 44L, to and through a middle region 44M, to a right region 44R. Of course, a sample may be introduced from a right region 44R and be drawn by capillary action to a middle region 44M and thence to a left region 44L.

In some embodiments, a second fluid may be similarly introduced at one side of a region similarly situated in an unlabeled region at the end of the one or more nano-channels opposite to that to which a sample may be introduced, and may be drawn by capillary action to a corresponding middle region, and thence be drawn by capillary action to a region on the side opposing the region into which said second fluid may be introduced.

In some embodiments, a sample may be introduced prior to introduction of a second fluid, such that said sample may be drawn into from a first end of one or more nano-channels, and through to a second end of said one or more nano-channels. The second fluid may thence be applied to a region adjacent to a middle region which intersects the second end of one or more nanochannels. In this way, an air gap or bubble may be prevented from forming between the sample and the second fluid in the one or more nano-channels as may occur if fluids were applied to both ends of the nanochannel simultaneously, thus permitting fluidic access through the one or more nano-channels. Similarly, the second fluid may be provided first, and drawn through the nano-channel by capillary action prior to introduction of a sample fluid.

Formation of such an air gap or bubble may be more likely to form when the distance between the ends of a nano-channel(s) is long relative to the width height or diameter, or other measurement associated with the cross section of a nano-channel(s). In some embodiments, a length of a nano-channel(s) may be 10 times the minimum dimension of the cross section, which may be the height, width or diameter of a nano-channel(s). In further embodiments, a length of a nano-channel(s) may be 100 times the minimum dimension of the cross section, which may be the height, width or diameter of a nano-channel(s).

In further embodiments, a length of a nano-channel may be longer than a sample DNA oligo such that said sample DNA oligo may fit completely within said nano-channel(s), wherein said sample DNA oligo may be from 100 to 200 bases in length, or may be from 150 to 500 bases in length, or may be from 300 to 1000 bases in length, or may be of 800 to 4000 bases in length, or may be from 3000 to 10,000 bases in length, or may be from 8,000 to 100,000 bases in length, or may be greater than 100,000 bases in length.

Furthermore, one or more flow directors 42, wherein each may extend towards the entrance to a nano-channel(s), such that the a width of the channel may be reduced in the region of the area of the channel immediately adjacent to the nano-channel(s) 52, optionally providing a flow director at end(s) of a nano-channel(s) 52 wherein a nano-gap electrode pair 12 may be located such that the flow directors 42 are oppositely arranged, or may be arranged such that one end of a nanochannel has an associated piece of insulation of flow controller, while the other end may not have such a feature. Flow director(s) 42 may serve to direct a flow such that a sample molecule may be brought close to one end of a nano-channel(s), so as to allow a higher percentage of sample molecules to be introduced into said nano-channel(s), and said sample molecules may be introduced more quickly. As such, two flow paths for the sample 50 are formed, i.e., a flow path 46A extending from the left region 44L to an inter-electrode region of the nano-gap electrode pair 12, and a flow path 46B extending from the left region 44L to an upper right region including the nano-pillars 40 as viewed in FIG. 2. In other words, flow director(s) 42 may serve to direct movement of sample 50 by forming various flow paths, including the flow path 46A for flowing the sample 50 in the direction towards the nano-gap electrode pair 12, and the flow path 46B for flowing the sample 50 in the direction away from the nano-gap electrode pair 12.

In some embodiments, nano-channel(s), first and second channels, pillars, and nanoelectrode pair(s) may be created lithographically on substantially the same plane.

When such flow director(s) 42 is not present, as conventionally, the flow path of the sample 50 is only directed in the arrow B direction, i.e., the direction toward the nano-gap electrode pair 12. Therefore, the sample 50 is likely to build up at a region near the nano-gap electrode pair 12 where the flow path narrows. In contrast, in some embodiments, the two flow paths 46A and 46B are formed by placing flow director(s) 42, so that excess sample 50 may flow to region 44R through flow path 46B. In this way, clogging of sample 50 near nano-gap electrode pair 12 may be reduced, so that high accuracy identification of the single molecule 52 is enabled.

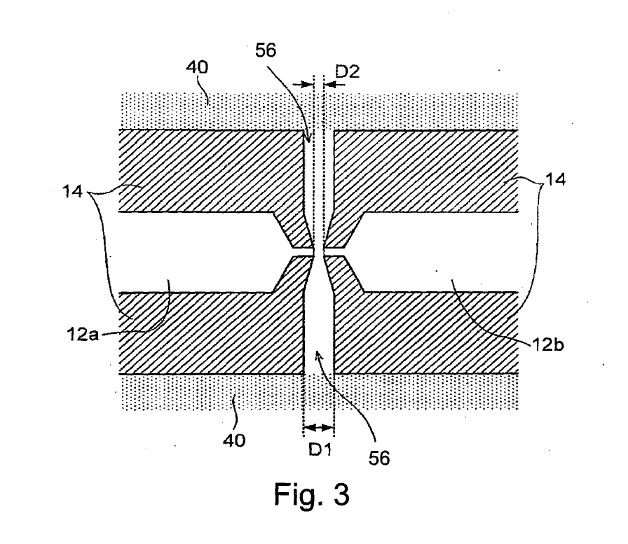

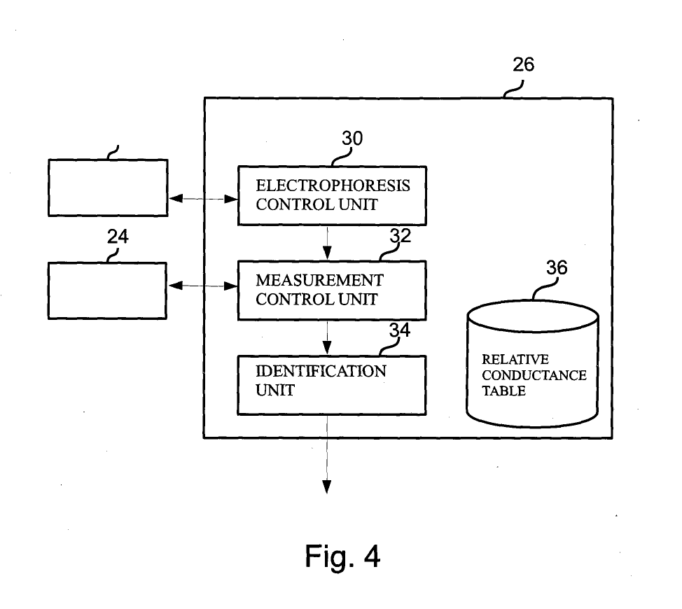

FIG. 3 is an enlarged view of region 54 delineated by a broken line in FIG. 2. As illustrated in FIG. 3, a nano-channel(s) 56 may be formed near to one or more insulators 14 and immediately adjacent to middle region 44M, which may be configured so as to be on opposite ends of nano-channel(s) 52. Nano-channel(s) 56 may have a tapered shape from middle region 44M, in which nano-pillars 40 may be provided, towards the electrodes of nano-gap electrode pair 12. Such tapering may permit a biomolecule to linearize upon flow through the nano-channel(s) 56. Width D1 of nano-channel(s) 56 at a position closer to middle region 44M may be about 120 nm, by way of example, but may be any appropriate width, such as 20 nm-100 nm, 50 nm-250nm, or 200 nm-1000 nm. As described above, it may be desirable that a width of nano-channel(s) 56 at a tapering point, that is, inter-electrode distance D2 of nano-gap electrode pair 12, may be slightly shorter than, equal to, or slightly longer than a molecule diameter of a single molecule 52. By way of example, a single molecule may have a molecular diameter of a few hundred picometers (pm) to 1.0 nm or more.

As illustrated in FIG. 2, an electrophoresis electrode pair 20 may be disposed in fluidic communication with the nano-channel(s) 56. This enables a consistent electrophoretic mobility for each single molecule and enables identification of a single molecule with high accuracy and high throughput.

System control unit 26 may control the respective components of a biomolecule sequencing device 10, and may identify the kind (or type) of target single molecule 52 based on a signal according to changes in a measured tunneling signal.

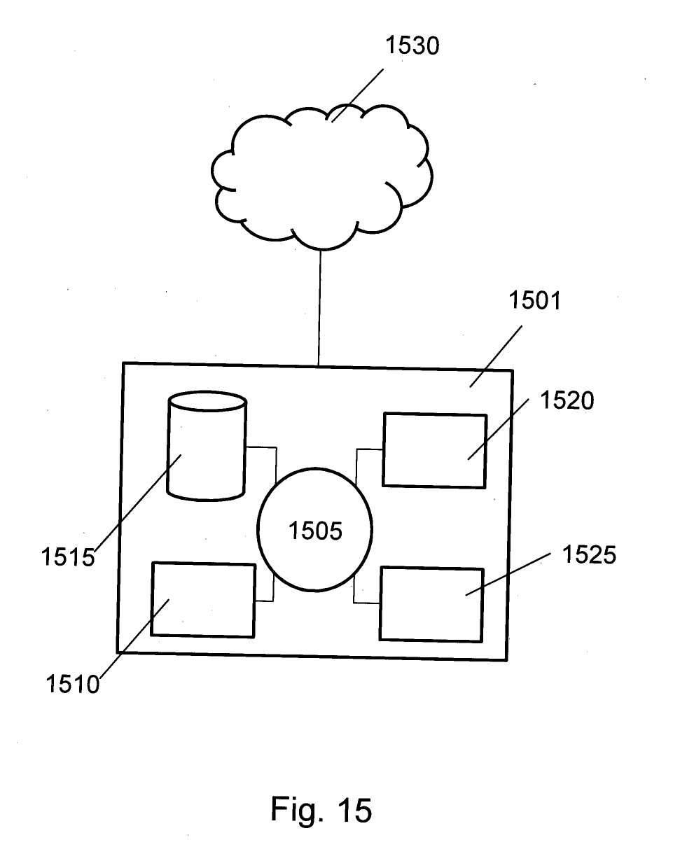

System control unit 26 may be a computer having a central processing unit (CPU), random access memory (RAM), read only memory (ROM) in which a biomolecule sequencing program (described later) may be stored, etc. The system control unit 26 may be as described elsewhere herein, such as FIG. 15 and the corresponding text. System control unit 26 may comprise a computer and may be functionally represented as including an electrophoresis control unit 30, a measurement control unit 32, and an identification unit 34. Hereafter, the respective components will be described in detail.

Electrophoresis control unit 30 may control application of voltage by one or more electrophoresis power supply devices 22 such that single molecule 52 may pass between the electrodes of a nano-gap electrode pair 12.

Measurement control unit 32 may control ammeter 24 to cause ammeter 24 to measure a tunneling current flowing between the electrodes of nano-gap electrode pair 12. There is no particular limit to the duration for measuring the tunnel current, and possible values thereof are 10 minutes, 20 minutes, 30 minutes, 40 minutes, 50 minutes 1, 2, 3, 4 or more hours. The time for measurement may be appropriately set according to the length of the single molecules 52, the number of single molecules to be sequenced, the error rate of sequencing, the coverage for which the single molecules are to sequenced, the number of nano-channels and sensors which are utilized for sequencing, among other factors.

Furthermore, measurement control unit 32 may obtain current value of tunneling current measured by ammeter 24, calculate conductance using an obtained current value, and prepare a conductance-time profile. Conductance may be calculated by dividing a current value of tunnel current by a voltage V that has been applied across a nano-gap electrode pair 12 when a tunnel current is measured. Using conductance enables the obtaining of a unified standard profile even if a voltage to be applied across a nano-gap electrode pair 12 is different for different measurements. When a voltage value to be applied across a nano-gap electrode pair 12 is made constant for each measurement, a current value of a tunnel current and a conductance may be treated in the same manner.

Measurement control unit 32 may use a current amplifier so as to amplify a tunneling current measured by ammeter 24 and obtain a so-amplified tunneling current. Using a current amplifier enables amplification of weak tunneling current values. Thus, measuring a tunnel current with high sensitivity is allowed. A current amplifier may be, for example, a commercially available variable-gain high-speed current amplifier (Catalogue Number: DHPCA-100, manufactured by FEMTO Messtechnik GmbH).

Identification unit 34 may compare detected physical quantity(s) obtained from a conductance-time profile prepared by measurement control unit 32 utilizing a relative conductance, relative to monomers in a single molecule 52 of which the kind (or type) is known, stored in a relative conductance table 36, thereby identifying a kind of target monomer in a single molecule 52. In some embodiments, a detected physical quantity may be a conductance for each measurement point of a conductance-time profile prepared by measurement control unit 32. As used herein, relative conductance is a conductance for each kind of monomer in a single molecule obtained by measuring a monomer in a single molecule 52 for which the kind is known. A relative conductance may be calculated by dividing a measured conductance associated with each monomer of a single molecule 52 by with a maximum measured conductance value measured for all of monomers in a single molecule 52. In some embodiments a measured conductance may be a maximum or modal conductance.

In some embodiments, at least one single molecule 52 to be identified may be dissolved in a solvent. There is no particular limitation to the solvent. For example, ultrapure water may be used. Ultrapure water can, for example, be produced using a Milli-Q® Integral 3 (device name) made by EMD Millipore Corporation (Milli-Q® Integral 3/5/10/15 (catalogue number)). The concentration of single molecules 52 in a solution is not particularly limited; however, it may be, for example, 0.01 to 1.0 μM, or 0.5 to 5.0 μM, or 2 to 20 μM, or 10 to 100 μM.

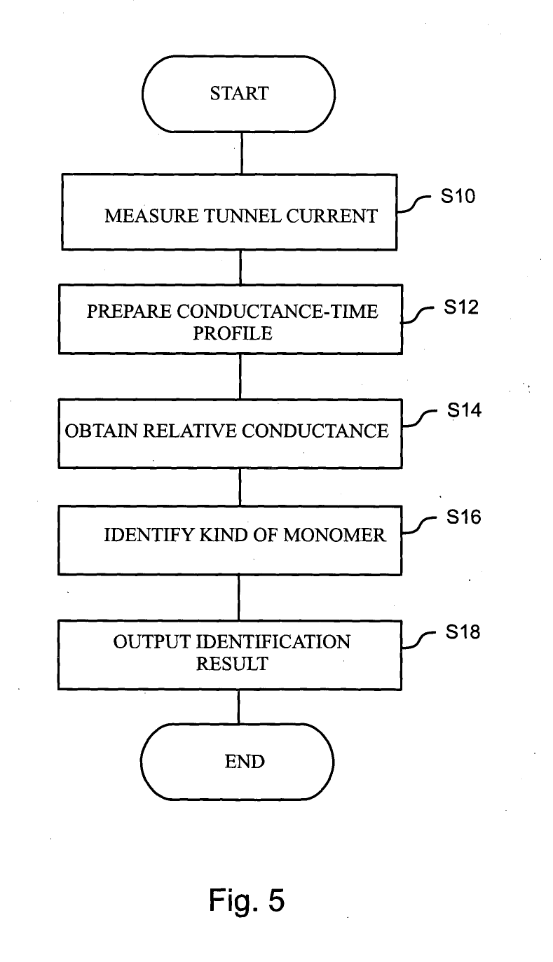

Then, nano-gap electrode pair(s) 12 may be immersed in the sample, measurement power supply device(s) 18 may be used to apply a voltage across nano-gap electrode pair(s) 12, and electrophoresis power supply device(s) 22 may be used to apply a voltage across electrophoresis electrode pair(s) 20. A CPU of a computer that constitutes a control unit which may read out and execute a biomolecule sequencing program stored in ROM or other nonvolatile storage. This may cause biomolecule sequencing device 10 to execute a biomolecule sequencing process as illustrated in FIG. 5.

In step S10, measurement control unit 32 may controls ammeter 24 so as to cause ammeter 24 to measure tunneling current that is generated when a single molecule 52 passes between the electrodes of a nano-gap electrode pair 12.

In step S12, measurement control unit 32 obtains current values of a measured tunneling current, calculates a conductance for each measurement point, and prepares a conductance-time profile.

In step S14, identification unit 34 obtains a relative conductance of different monomers of target single molecule 52 from relative conductance table 36.

In step S16, identification unit 34 may compare a conductance-time profile prepared in step S12 above with a relative conductance obtained in step S14 above, and identify the kind of monomer indicated by each signal. In step S18, identification unit 34 may output an identification result. Thus, a monomer identification process for a single molecule may be completed.

As described above, in some embodiments of a biomolecule sequencing device as described herein above, electrophoresis electrode pair 20 may be disposed near a nano-channel 56, and flow path 46A for moving sample 50 in the direction through nano-channel(s) 56 and towards nano-gap electrode pair(s) 12, and flow path 46B for flowing the sample 50 in the direction past the entrance to nano-channel(s) 56 in which may contain one or more nano-gap electrode pair(s) 12.

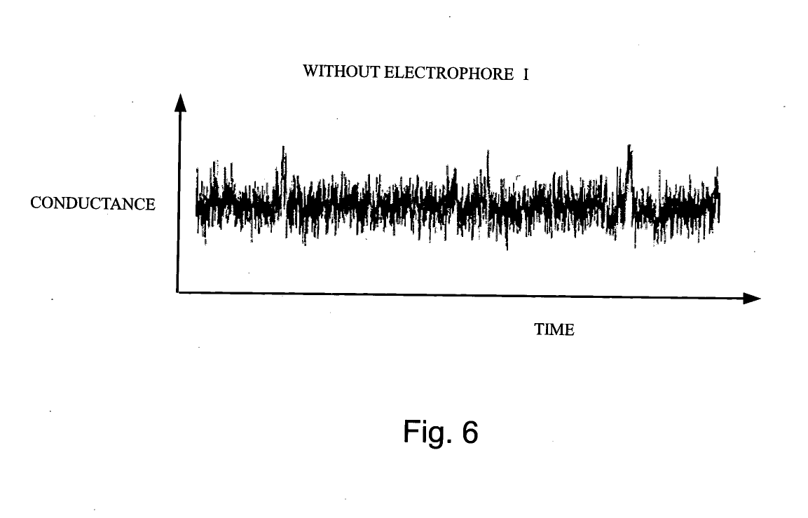

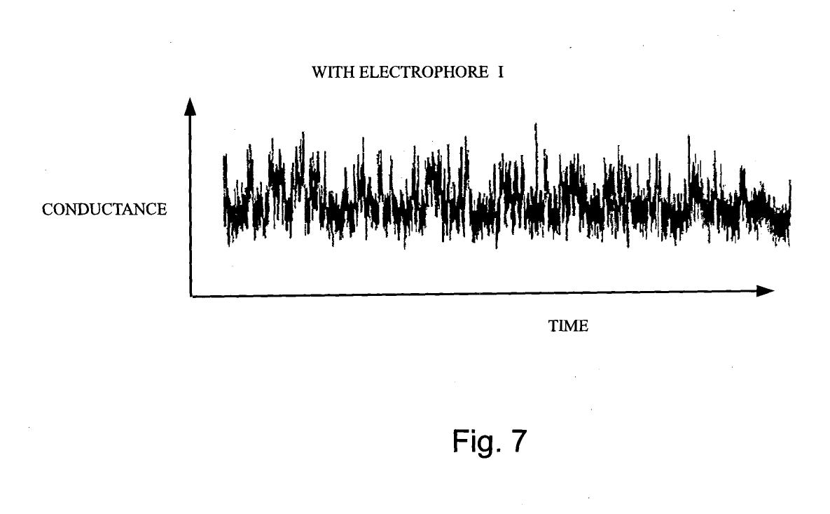

This enables a high signal frequency based on measurement of tunneling current passing between the electrodes of nano-gap electrode pair(s) 12. FIG. 6 illustrates a signal waveform detected by a device in which no electrophoresis electrode pair 20 is provided in a conventional manner, thus only utilizing Brownian motion, and FIG. 7 illustrates a signal waveform detected by a device in which an electrophoresis electrode pair 20 is provided as in biomolecule sequencing device 10 in some present embodiments. FIGS. 6 and 7 are shown with the same scale for both conductance and time. As can be seen, FIG. 7 has more periods of time (pulses) with increased conductance. The increased conductance is associated with the presence of DNA in the nano-electrode pair gap. As can be seen in FIGS. 6 and 7, it is understood that biomolecule sequencing device 10 in some embodiments exhibits a high signal frequency compared with a conventional manner.

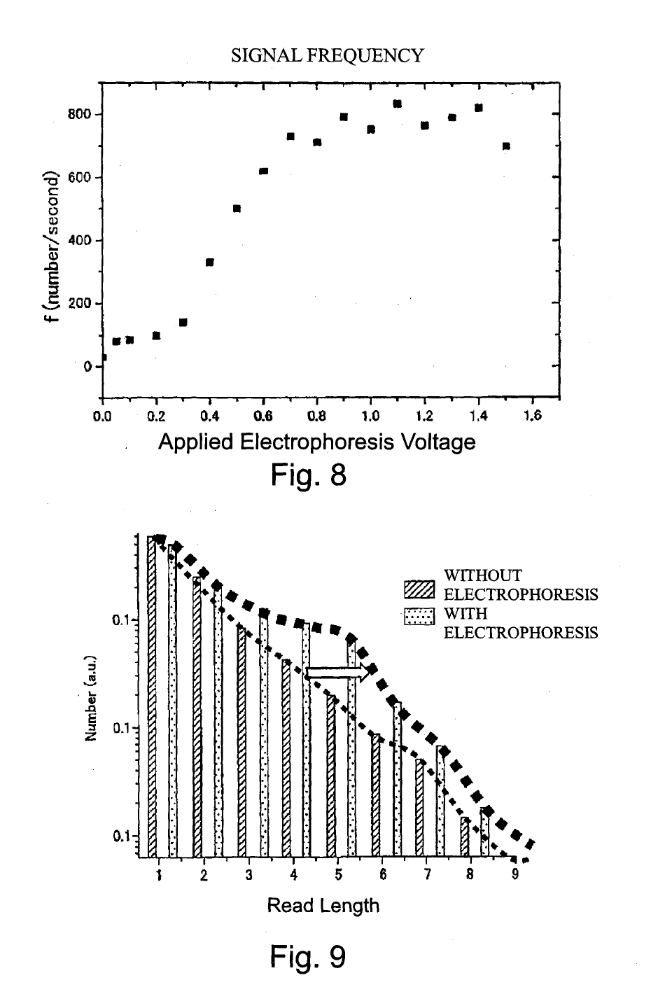

FIG. 8 is a graph showing the results of measuring the relationship between a voltage applied to an electrophoresis electrode pair 20 and the number of signals detected per second (signal frequency). As can be seen in FIG. 8, it is understood that in this exemplary configuration, until the voltage applied to electrophoresis electrode pair 20 increases to about 0.7 V, the signal frequency increases with an increase in the electrophoresis voltage.

FIG. 9 shows measurement results of the numbers of fragments read of different fragment read lengths for a plurality of different kinds of single molecules, when electrophoresis electrode pair 20 with a voltage applied thereto is provided and when the electrophoresis electrode pair 20 is not provided. As can be seen from FIG. 9, it is understood that the number of reads when an electrophoresis electrode pair 20 with a voltage applied thereto is provided is large, compared with when an electrophoresis electrode pair 20 is not provided.

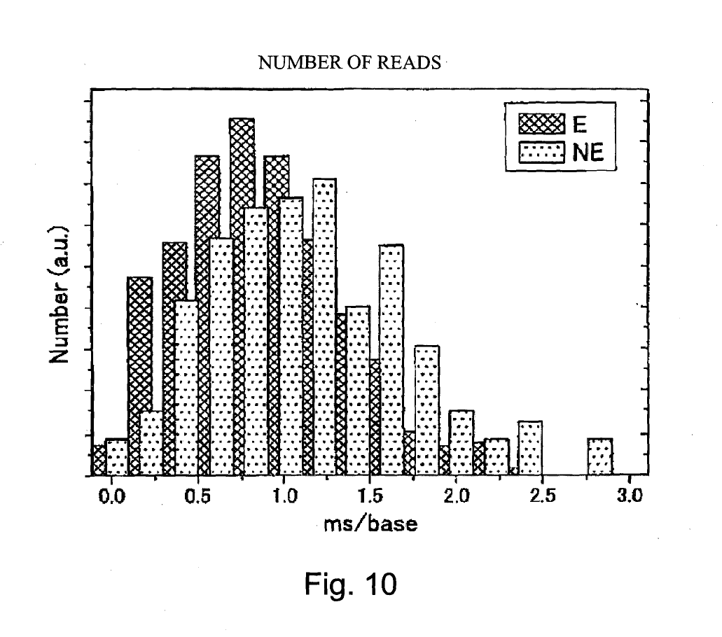

FIG. 10 shows the results of measuring the number of reads different read lengths, when an electrophoresis electrode pair 20 is not provided (NE) and when an electrophoresis electrode pair 20 is provided with a voltage applied thereto (N). As can be seen from FIG. 10, it is understood that the number of reads when an electrophoresis electrode pair 20 is provided with a voltage applied thereto is larger relative to a system wherein a electrophoresis electrode pair is not supplied (NE) in the range of 1.0 ms/base or less.

Table 1 below shows the results of measuring the average number of reads and the maximum number of reads for a single molecule, signal frequencies, and necessary volume of sample. Signal frequency is measured when the sample 50 concentration is 10-7 M (mole).

TABLE 1

| | | Present | Present Invention/ |
|---|---|---|---|
| | Conventional | Invention | Conventional |
| Average Number of Reads | 1.4 (12) | 2.2 (18) | About 1.5 times |
| (Maximum Number of | | | |
| Reads) | | | |
| Signal Frequency | 0 to 30/sec | 500/sec | About 16 times |
| | | | or more |
| Necessary Volume | 10 to 20 μl | 1 to 2 μl | About 1/10 |
| of Sample | | | |

As shown in Table 1, it is understood that the present invention is superior in all of the average and maximum number of reads for single molecules, signal frequency, and lower volume of a sample needed, compared with the conventional art.

As described above, a biomolecule sequencing device in some embodiments may be configured so that electrophoresis electrode pair 20 may be disposed near nano-gap electrode pair 12, and both flow path 46A for flowing a sample 50 in the direction into a nano-channel(s) 56 towards nano-gap electrode pair(s) 12 and flow path 46B for flowing a sample 50 in the direction past a nano-channel(s) 56 which may contain nano-gap electrode pair(s) 12, so that an increase in the electric field impressed upon a single molecule 52 can be improved. This may enable greater stabilization of passing speed relative to Brownian motion, of a single molecule relative to one or more nano-electrode pair(s). This enables longer reads and identification of single molecules with high accuracy and high throughput.

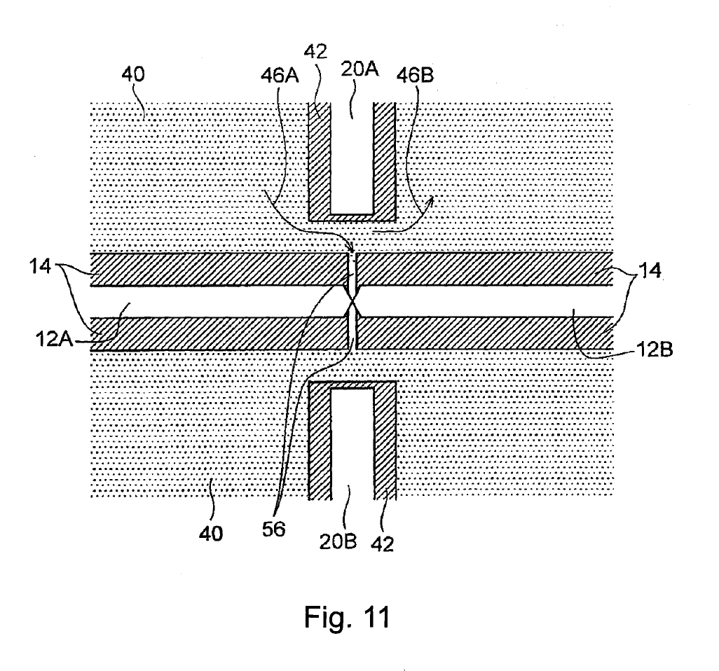

In some embodiments, a structure in which an electrophoresis electrode pair 20 is arranged in parallel with the nano-gap electrode pair 12 is explained. However, as illustrated in FIG. 11, an electrophoresis electrode pair 20 may be disposed so that the electrodes thereof are disposed on or immediately adjacent to flow director(s) 42. In other words, electrophoresis electrode pair 20 may be extended close to nano-channel(s) 56 which may contain electrode pair(s) 12 along the direction of introducing sample 50. In this case, electrodes of an electrophoresis electrode pair 20 may be respectively disposed just above and just below a nano-channel(s) 56. This enables further increase of the electric field impressed upon a single molecule 52, and improves identification of monomers of single molecules with high accuracy and high throughput.

Next, additional embodiments of the invention will be described wherein multiple different nano-gap spacings may be utilized. Components or parts corresponding to or similar to those of the biomolecule sequencing device 10 of FIG. 1 are designated by the same reference numerals, and explanation thereof will be omitted.

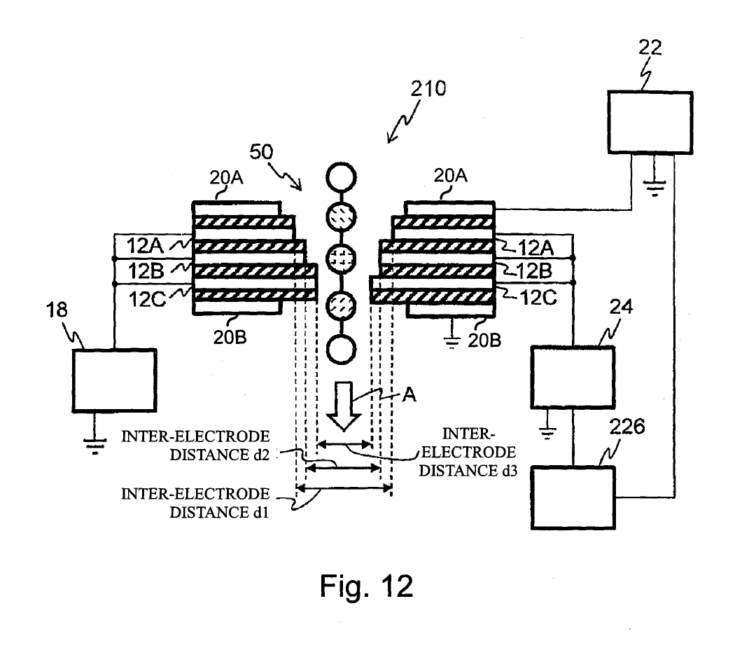

As illustrated in FIG. 12, a biomolecule sequencing device 210 according to some embodiments includes nano-gap electrode pairs 12A, 12B and 12C, one or more measurement power supply devices 18 for measurement, one or more electrophoresis electrode pairs 20, one or more electrophoresis power supply devices 22 for electrophoresis, an ammeter 24, and one or more system control units 226.

The structure of nano-gap electrode pairs 12A, 12B and 12C may be the same as that of nano-gap electrode pair(s) 12 as described with respect to FIG. 1. The electrodes of each nano-gap electrode pair 12A, 12B and 12C may be aligned so that the center lines between electrode pairs are aligned on the same axis. In other words, a single path through which a single molecule 52 passes may be defined in part by the inter-electrode spaces of nano-gap electrode pairs 12A, 12B, and 12C. The inter-electrode distance of nano-gap electrode pair 12A may be d1, the inter-electrode distance of nano-gap electrode pair 12B may be d2, and the inter-electrode distance of nano-gap electrode pair 12C d3 may be different from one another. In the example illustrated in FIG. 12, the relationship thereof is d1>d2>d3. For example, these distances may be d1=1.0 nm, d2=0.7 nm, d3=0.5 nm, but may be any distance as needed for an application, for example if it is desired to measure amino acids, one inter-electrode distance may be 0.25 nm, while another inter-electrode distance may be greater than 1.0 nm, each being less than 20% less than a molecular diameter for a side chain for different amino acids. In some embodiments some of the inter-electrode distances may be the same or substantially the same.

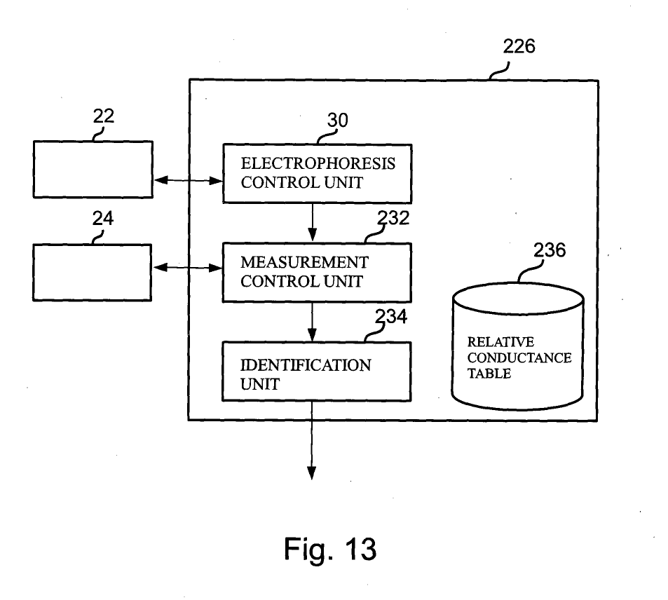

As illustrated in FIG. 13, system control unit 226 may be represented as a system including an electrophoresis control unit 30, a measurement control unit 232, and an identification unit 234.

Measurement control unit 232 may control ammeter 24 to cause ammeter 24 to measure each tunneling currents generated between nano-gap electrode pairs 12A, 12B, and 12C. Measurement control unit 232 may also obtain current values of tunneling current for each inter-electrode distance measured by ammeter 24, calculate a conductance, and prepare a conductance-time profile for each inter-electrode distance.

Identification unit 234 may compare detected physical quantities obtained from a conductance-time profile for each inter-electrode distance as determined by measurement control unit 32 with relative conductance, stored in a relative conductance table 236, regarding monomers of a single molecule 52 of which the kind is known, thereby identifying the kind of monomers of a target single molecule 52.

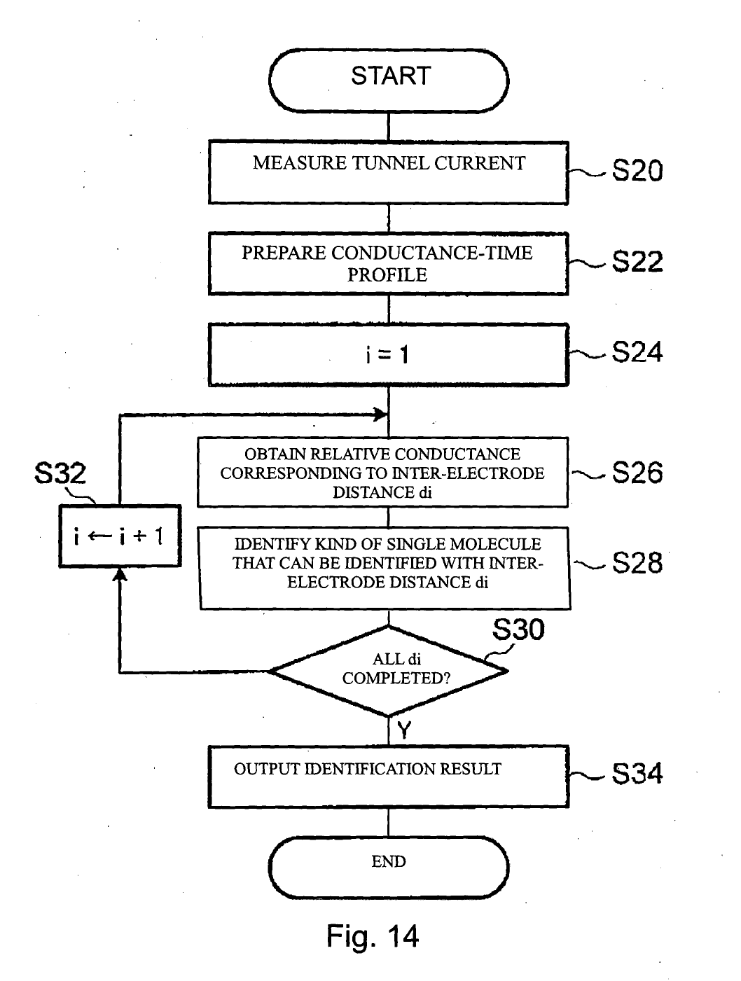

In some embodiments, at least one single molecule 52 to be identified may be dissolved in a solvent as described hereinabove. Then, nano-gap electrode pairs 12A, 12B and 12C may be immersed in the sample, measurement power supply device(s) 18 may be used to apply a voltage across each of nano-gap electrode pairs 12A, 12B and 12C, and electrophoresis power supply device(s) 22 may be used to apply a voltage across electrophoresis electrode pair 20A and 20B. A CPU of a computer that constitutes system control unit 226 may read out and execute a biomolecule sequencing program which may be stored in the ROM or other nonvolatile memory. This may cause biomolecule sequencing device 210 to execute a biomolecule sequencing process as illustrated in FIG. 14.

The nano-gap electrode pairs 12A, 12B and 12C can have different gap sizes. The gap sizes can be selected to permit the identification of different types of monomers (or subunits) of a biomolecule. For example, nano-gap electrode pair 12A can have a width that is selected to permit the identification of one type of nucleotide (e.g., adenosine) and the nano-gap electrode pair 12B can have a width that is selected to permit the identification of another type of nucleotide (e.g., thymine).

The nano-gap electrode pairs 12A, 12B and 12C can be situated in a nano-channel. The nano-channel can include any number of nano-gap electrode pairs, such as at least 2, 3, 4, 5, 6, 7, 8, 9, or 10 nano-gap electrode pairs. At least some or all of the nano-gap electrode pairs can have different widths.

In step S20, measurement control unit 232 controls ammeter 24 so as to cause ammeter 24 to measure a tunneling current generated when single molecule 52 passes through nano-channel(s) 56 formed in part between the electrodes of nano-gap electrode pairs 12A, 12B and 12C.

In step S22, measurement control unit 232 may obtain current values of a measured tunneling current, calculate conductance for each measurement point, and prepare a conductance-time profile for each inter-electrode distance.

In step S24, identification unit 234 may set variable “i” to 1.

In step S26, identification unit 234 may obtain a relative conductance of a monomer of a single molecule 52 corresponding to inter-electrode distance dᵢ, i.e., the relative conductance of monomers of target single molecules 52 that can be identified with an inter-electrode distance dᵢ.

In step S28, identification unit 234 compares conductance-time profiles of inter-electrode distance dᵢ prepared in step S22 above with relative conductance values obtained in step S26 above, and identifies a kind of monomer of a single molecule indicated by each signal.

In step S30, identification unit 234 may determines whether a process has been completed for all inter-electrode distances dᵢ. If there is an unprocessed inter-electrode distance dᵢ, the process proceeds to step S32, and increments “i” by 1, and returns to step S26. When the process has been completed for all inter-electrode distances d₁, then the process proceeds to step S34, identification unit 234 may output an identification result, and the biomolecule sequencing process is completed.

As described hereinabove, in some embodiments, conductance may be used that is obtained utilizing current (e.g., tunneling current) generated between a plurality of nano-gap electrode pairs that differ in inter-electrode distance. Accordingly, in addition to the advantage effects effectuated by use of a single nano-gap electrode pair or several nano-gap electrode pairs with the same or essentially similar nano-gap spacings, more accurate identification is enabled. Not only a structure in which a plurality of nano-gap electrode pairs that are different in inter-electrode distance, but also a structure in which an inter-electrode distance of a single nano-gap electrode pair can be changed, are possible.

In the some embodiments as described herein, only the case for which a plurality of nano-gap electrode pairs that differ in inter-electrode distance has been explained. However, a structure in which an inter-electrode distance for a single nano-gap electrode pair is changed is possible. For example, an inter-electrode distance may be changed by adjusting the geometrical arrangement of the point of effort, a pivot point, and the point of load, using principles of leverage. More specifically, the inter-electrode distance may be changed by moving an end of an electrode that serves as the point of load by pushing up a part of a nano-gap electrode pair using piezoelectric elements. In this case, an inter-electrode distance can be set as desired based on a correspondence relationship between a distance pushed up by piezoelectric element(s) and an inter-electrode distance.

A biomolecule sequencing device may be configured so that electrophoresis electrode pair 20 may be disposed near nano-gap electrode pair 12, and both flow path 46A for flowing a sample 50 in the direction into nano-channel(s) 56 towards nano-gap electrode pair(s) 12 and flow path 46B for flowing a sample 50 in a direction past nano-channel(s) 56 in which nano-gap electrode pair 12 may be provided, so that an increase of the electric field seen by a single molecule 52 can be improved. This enables identification of a single molecule with high accuracy and high throughput. Furthermore, in some embodiments a biomolecule sequencing device may be used as a proteomic sequencer, and can be applicable, for example, to allergen tests, disease diagnosis, etc., with high speed, high sensitivity, and low cost, used in the fields of public health, safety, security, and the environment.

While the present disclosure has made reference to a “pair” of nano-gap electrodes, it will be appreciated that devices and systems of the present disclosure can include any number of nano-gap electrodes. For example, a device can include a set of nano-gap electrodes, with the set including at least 2, 3, 4, 5, 6, 7, 8, 9, or 10 electrodes.

### Computer Control Systems

The present disclosure provides computer control systems that are programmed to implement methods of the disclosure. FIG. 15 shows a computer system 1501 that is programmed or otherwise configured to sequence a biomolecule, such as a protein. The computer system 1501 can be the control units 26 and 226 described elsewhere herein. The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine (or computer) readable medium, such as computer-executable code (or computer program), may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

As described hereinabove, in some embodiments the invention biomolecule sequencing system is described as comprising a program that has been preinstalled. However, a program stored in an external memory or external recording medium, etc., may be read or downloaded via the internet at any time for execution. Furthermore, this program may be provided in a state stored in a computer readable recording medium.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

This application claims priority to Japanese Patent Application No. 2014-011430, filed Jan. 24, 2014, which is entirely incorporated herein by reference.

All publications, patents, and patent applications mentioned in this specification are herein incorporated by reference to the same extent as if each individual publication, patent, or patent application was specifically and individually indicated to be incorporated by reference.

1. A biomolecule sequencing device, comprising:
a nano-channel that permits a sample containing a biomolecule to move through the nano-channel;
a plurality of sets of nano-gap electrodes in the nano-channel, wherein each set of the plurality of nano-gap electrodes is configured to permit the detection of a current when the biomolecule contained in the sample passes through the nano-channel and in proximity to the plurality of sets of nano-gap electrodes, and wherein at least two sets of the plurality of sets of nano-gap electrodes have different inter-electrode distances along a width of the nano-channel; and
a set of electrophoresis electrodes that provide an electric field to subject the biomolecule to motion through the nano-channel and in proximity to the plurality of sets of nano-gap electrodes in the nano-channel.

2. The biomolecule sequencing device of claim 1, further comprising:
a measurement unit in communication with each of the plurality of sets of nano-gap electrodes, wherein the measurement unit is configured to measure the current generated when the biomolecule passes in proximity to the plurality of sets of nano-gap electrodes; and
an identification unit in communication with the measurement unit, wherein the identification unit is configured to identify the biomolecule or a portion thereof.

3. The biomolecule sequencing device of claim 2, wherein the biomolecule includes a plurality of monomers, and wherein the identification unit is configured to identify the plurality of monomers based on a reference physical quantity of at least one known type of monomer and a physical quantity obtained from the current measured by the measurement unit.

4. The biomolecule sequencing device of claim 1, further comprising a flow director configured to generate a first flow path and a second flow path that are in fluid communication with the nano-channel, wherein the flow director directs a portion of the sample from the first flow path to the nano-channel and a remainder of the sample from the first flow path to the second flow path.

5. The biomolecule sequencing device of claim 4, wherein the flow director is an insulator that extends towards the plurality of sets of nano-gap electrodes along a direction of movement of the sample through the nano-channel.

6. The biomolecule sequencing device of claim 4, further comprising one or more pillars in the first flow path and/or the second flow path to permit linearization of the biomolecule.

7. (canceled)

8. The biomolecule sequencing device of claim 4, wherein the first flow path, the second flow path and the nano-channel are substantially in the same plane.

9. The biomolecule sequencing device of claim 1, wherein the current includes tunneling current.

10. (canceled)

11. (canceled)

12. The biomolecule sequencing device of claim 1, wherein the plurality of sets of nano-gap electrodes and the set of electrophoresis electrodes are integrated as a single-piece unit.

13. The biomolecule sequencing device of claim 12, wherein electrodes of a given set of the plurality of sets of nano-gap electrodes are separated from the electrophoresis electrodes by at least one solid state insulator.

14. The biomolecule sequencing device of claim 1, further comprising one or more pillars in the nano-channel to permit linearization of the biomolecule.

15. (canceled)

16. The biomolecule sequencing device of claim 1, wherein the nano-channel is tapered towards the plurality of sets of nano-gap electrodes.

17. The biomolecule sequencing device of claim 1, wherein a given set of the plurality of sets of nano-gap electrodes has an inter-electrode distance that is less than or equal to a molecular diameter of the biomolecule.

18. (canceled)

19. (canceled)

20. A method for sequencing a biomolecule, comprising:
(a) directing the biomolecule to flow to or through a nano-channel of a biomolecule sequencing device, wherein the biomolecule sequencing device includes (i) a plurality of sets of nano-gap electrodes in the nano-channel, wherein each set of the plurality of nano-gap electrodes is configured to permit the detection of a current when the biomolecule contained in the sample passes through the nano-channel and in proximity to the plurality of sets of nano-gap electrodes, and wherein at least two sets of the plurality of sets of nano-gap electrodes have different interelectrode distances along a width of the nano-channel, and (ii) a set of electrophoresis electrodes that provide an electric field to subject the biomolecule to motion to or through the nano-channel and in proximity to the plurality of sets of nano-gap electrodes in the nano-channel;
(b) with the plurality of sets of nano-gap electrodes, detecting current generated while the biomolecule flows through the nano-channel and in proximity to the plurality of sets of nano-gap electrodes; and
(c) sequencing the biomolecule or a portion thereof with the current detected in (b).

21. The method of claim 20, wherein the biomolecule includes a plurality of monomers, and wherein the sequencing comprises identifying the plurality of monomers based on a reference physical quantity of at least one known type of monomer and a physical quantity obtained from the current detected in (b).

22. The method of claim 20, wherein the biomolecule sequencing device further comprises a flow director configured to generate a first flow path and a second flow path that are in fluid communication with the nano-channel, and wherein (a) comprises flowing a portion of the sample from the first flow path to the nano-channel and a remainder of the sample from the first flow path to the second flow path.

23. The method of claim 22, further comprising one or more pillars in the first flow path and/or the second flow path to permit linearization of the biomolecule.

24. The method of claim 20, wherein the current includes tunneling current.

25. The method of claim 20, further comprising one or more pillars in the nano-channel that linearize the biomolecule.

26. The method of claim 20, wherein the nano-channel is tapered towards the plurality of sets of nano-gap electrodes.

27. The method of claim 20, wherein the biomolecule is a polynucleotide or a polypeptide.

28. (canceled)

29. (canceled)

30. A computer readable medium comprising machine executable code that upon execution by one or more computer processors implements a method for sequencing a biomolecule, comprising:
(a) directing the biomolecule to flow to or through a nano-channel of a biomolecule sequencing device, wherein the biomolecule sequencing device includes (i) a plurality of sets of nano-gap electrodes in the nano-channel, wherein each set of the plurality of nano-gap electrodes is configured to permit the detection of a current when the biomolecule contained in the sample passes through the nano-channel and in proximity to the plurality of sets of nano-gap electrodes, and wherein at least two sets of the plurality of sets of nano-gap electrodes have different interelectrode distances along a width of the nano-channel, and (ii) a set of electrophoresis electrodes that provide an electric field to subject the biomolecule to motion to or through the nano-channel and in proximity to the plurality of sets of nano-gap electrodes in the nano-channel;
(b) with the plurality of sets of nano-gap electrodes, detecting current generated while the biomolecule flows through the nano-channel and in proximity to the plurality of sets of nano-gap electrodes; and
(c) sequencing the biomolecule or a portion thereof with the current detected in (b).

31. (canceled)

32. (canceled)
